(19) United States
(12) Patent Application Publication
(10) Pub. No.: US 2026/0201259 A1
(43) Pub. Date: Jul. 16, 2026

(54) Circular Products Produced by Feed Segregation to Parallel Cracking Units and Integrated Separation System

(71) Applicant: Chevron Phillips Chemical Company LP, The Woodlands, TX (US)

(72) Inventors: Jacob M. Hilbrich, Cleveland, TX (US); Scott G. Morrison, Kingwood, TX (US); Miguel Gonzalez Borja, Spring, TX (US)

(21) Appl. No.: 19/447,779

(22) Filed: Jan. 13, 2026

Related U.S. Application Data
(60) Provisional application No. 63/745,535, filed on Jan. 15, 2025.

(51) Int. Cl. C10G 67/02; B01D 3/14; B01J 6/00; B01J 8/18; B01J 12/00; B01J 19/24; C07C 2/06; C07C 4/04; C07C 4/06; C07C 5/03; C07C 7/00; C07C 7/04; C08F 110/02; C08F 110/06; C10G 1/02; C10G 11/18

(52) U.S. Cl. CPC C10G 67/02; B01D 3/143; B01J 6/008; B01J 8/1827; B01J 12/00; B01J 19/2445; B01J 19/245; C07C 2/06; C07C 4/04; C07C 4/06; C07C 5/03; C07C 7/005; C07C 7/04; C08F 110/02; C08F 110/06; C10G 1/02; C10G 11/18; C10G 2300/1007; C10G 2300/1011; C10G 2300/1062; C10G 2300/4081; C10G 2400/20

(57) ABSTRACT

Systems and processes for the production of circular products, including circular olefins and circular products derived from the circular olefins, by feeding particular hydrocarbons derived from one or more feed source to steam cracking and to fluid catalytic cracking and by sending the product of steam cracking and the product of fluid catalytic cracking to an integrated separation system. The circular olefins can be used in any downstream process to produce any circular product, such as a circular product produced through polymerization, oligomerization, or hydrogenation of the circular olefins.

### CROSS REFERENCE TO RELATED APPLICATIONS

This application claims benefit of U.S. Provisional Application No. 63/745,535 filed Jan. 15, 2025, and entitled “Circular Products Produced by Feed Segregation to Parallel Cracking Units and Integrated Separation System,” which is incorporated herein by reference in its entirety.

### FIELD OF THE DISCLOSURE

The present disclosure generally relates to the production of circular products, including circular olefins and circular products derived from the circular olefins, by sending products of fluid catalytic cracking and steam cracking to an integrated separation system.

### BACKGROUND

Steam cracking units and fluid catalytic cracking units are usually operated to convert larger hydrocarbon molecules into smaller hydrocarbon molecules. Each has benefits for cracking hydrocarbons into smaller molecules and/or olefins, depending on the type of feedstock fed to the respective unit. The product of steam cracking can flow to a quench tower for cooling and initial separation of the cracked product, and the product of fluid catalytic cracking can flow to a fractionation tower for separations into various product fractions.

Recovery of circular olefins for the production of circular chemicals or products from steam cracking units and fluid catalytic cracking units has been a challenge with regard to yield and conversion of feedstocks to commercially acceptable amounts of circular olefins.

A technology that can convert feedstocks such as waste plastics to olefins with a yield that is higher than currently available techniques, leading to a circular production of olefins and products derived from those circular olefins, is desirable.

### SUMMARY

Disclosed is a process that can include: cracking C2 hydrocarbons and C3 hydrocarbons in a steam cracking unit to form a cracked gas; cracking C4+ hydrocarbons in a fluid catalytic cracking unit to form a fluid catalytic cracking (FCC) product, wherein the C4+ hydrocarbons include butanes, butenes, naphtha, a pyrolysis oil, a used lubricating oil, a bio-based feedstock, a renewable feedstock, a non-renewable feedstock, a circular feedstock, or a combination thereof, or a combination thereof; introducing the cracked gas and the FCC product to a separation system; and recovering a plurality of hydrocarbon products from the separation system, wherein the plurality of hydrocarbon products includes one or more circular olefin.

Disclosed is a circular olefin system including: a steam cracking unit that cracks C2 hydrocarbons and C3 hydrocarbons into a cracked gas; a fluid catalytic cracking unit that cracks C4+ hydrocarbons into a fluid catalytic cracking (FCC) product, wherein the C4+ hydrocarbons include butanes, butenes, naphtha, a pyrolysis oil, a used lubricating oil, a bio-based feedstock, a renewable feedstock, a non-renewable feedstock, a circular feedstock, or a combination thereof; and a separation system coupled to the steam cracking unit and to the fluid catalytic cracking unit, wherein the separation system receives the cracked gas and the FCC product, wherein the separation system produces a plurality of hydrocarbon products including one or more circular olefin.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIG. 1 is a schematic diagram of a system for circular olefin production in which the disclosed processes can be performed.

FIG. 2 is a schematic diagram of the system of FIG. 1, having a first embodiment for the separation system.

FIG. 3 is a schematic diagram of the system of FIG. 1, having a second embodiment for the separation system.

FIG. 4 is a schematic diagram of the system of FIG. 1, having a third embodiment for the separation system.

FIG. 5 is a schematic diagram of an embodiment for the olefin recovery train that can be utilized in any embodiment of the separation system disclosed herein.

### DETAILED DESCRIPTION

The term “hydrocarbon” refers to a compound containing only carbon and hydrogen atoms.

The term “alkane” or “paraffin” refers to a saturated hydrocarbon compound.

The term “olefin” refers to hydrocarbons that have at least one carbon-carbon double bond, and includes linear and branched molecules.

The term “circular” refers to chemicals and products that can be certified in accordance with the International Sustainability and Carbon Certification (ISCC) provisions, as circular chemicals and products. Moreover, this disclosure demonstrates how chemicals and products may be certified as circular at any point along complex chemical reaction pathways disclosed herein.

Disclosed herein are systems and processes for the production of circular products by segregating or directing feeds derived from one or more feed sources to a steam cracking unit and to fluid catalytic cracking unit in the particular manner described herein and then sending the product of steam cracking and the product of catalytic cracking to an integrated separation system (the same separation system). That is, steam cracking and fluid catalytic cracking processes receive feed streams containing hydrocarbons of the particular carbon numbers disclosed herein and are operated in parallel, with the product of each cracking process sent to the same separation system for recovery of circular olefins for production of circular products from those circular olefins. The circular olefins can be used in any downstream process to produce any circular product, such as the polymerization or oligomerization described herein. It has been found that segregating feeds by feeding C2 and C3 hydrocarbons to the steam cracking unit and feeding one or more of butanes, butenes, naphtha, a pyrolysis oil, a used lubricating oil, a bio-based feedstock (e.g., vegetable oil), a renewable feedstock (e.g., bio-based renewable feedstock such as oils derived from algae, plants, or agricultural waste), a non-renewable feedstock (e.g., crude oil, NGLs), a circular feedstock (e.g., a circular pyrolysis oil derived from waste plastics), or a combination thereof, to the fluid catalytic cracking unit minimize the overall intensity of cracking these components. Then, by sending the products of these cracking processes to the same separation system (referred to herein as an integrated separation system), the production of circular olefins is maximized to favor production of circular products.

A further benefit of the disclosed processes and systems is a reduced carbon footprint per kg of product compared to a process or system that feeds all feed components only to a steam cracker.

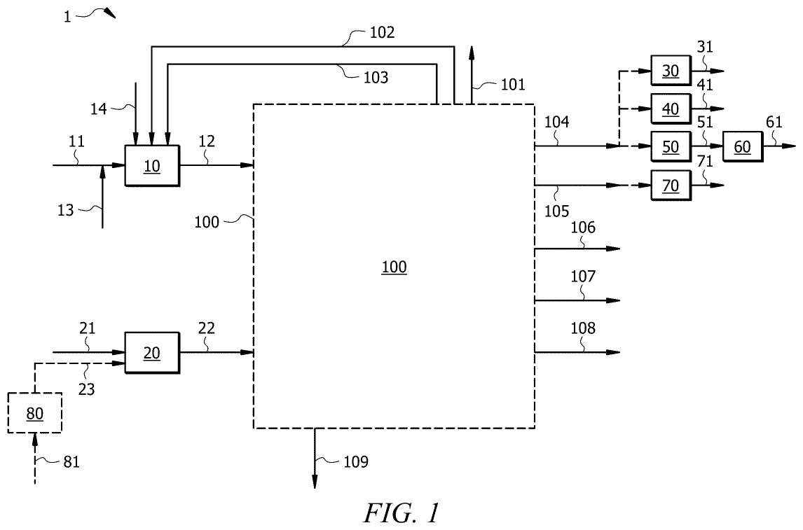

FIG. 1 is a schematic diagram of a system 1 for circular olefin production disclosed herein. The system 1 can include a steam cracking unit 10, a fluid catalytic cracking unit 20, and a separation system 100 coupled to the steam cracking unit 10 and to the fluid catalytic cracking unit 20. The system 1 can further include an ethylene polymerization reactor 30 coupled to the separation system 100, an oligomerization reactor 40 coupled to the separation system 100, a polyalphaolefin production system comprising an oligomerization reactor 50 coupled to the separation system 100 and a hydrogenation reactor 60 coupled to the oligomerization reactor 50, a propylene polymerization reactor 70 coupled to the separation system 100, or a combination thereof. In some embodiments, the system 1 can further include a pyrolysis unit 80 coupled to an inlet of the fluid catalytic cracking unit 20.

In the system 1, the separation system 100 receives a cracked gas 12 from the steam cracking unit 10 and a fluid catalytic cracking product (FCC product) 22 from the fluid catalytic cracking unit 20, and the separation system 100 produces hydrocarbon products in streams 101, 102, 103, 104, 105, 106, 107, 108, and 109.

### Steam Cracking Unit

The steam cracking unit 10 can be embodied with a conduit that passes C2 and C3 hydrocarbons from stream 11 through the interior of a fuel fired furnace at a temperature sufficient to convert one or more alkanes in the C2 and C3 hydrocarbons to one or more olefin (e.g., ethane to ethylene, propane to propylene, or both). The conduit can be configured to pass through a furnace housing where a fuel gas is combusted in the presence of oxygen to produce heat for cracking of the hydrocarbons in the conduit. Steam is introduced into the conduit via stream 13 for steam cracking of the hydrocarbons. Air or oxygen can be introduced into the fuel fired furnace via stream 14 for combustion of the fuel gas. The furnace housing can have burners configured to provide flames for combustion of fuel gas that is received into the furnace of the steam cracking unit 10. In aspects, the burners are configured with metallurgy for hydrocarbon-based combustion in the furnace housing. The combustion products of the furnace can flow from the steam cracking unit 10 as a flue gas. In aspects, the fuel gas can include an ethane product in stream 102 and/or propane product in stream 103 received from the separation system 100.

The feed stream 11 for the steam cracking unit 10 can include hydrocarbons having from 2 to 3 carbon atoms. In aspects, the feed stream 11 consists of C2 and C3 hydrocarbons, and only C2 hydrocarbons and C3 hydrocarbons are fed to the steam cracking unit 10. In additional or alternative aspects, the feed stream 11 does not include naphtha hydrocarbons. In aspects, no naphtha is fed to the steam cracking unit 10.

In aspects, the feed stream 11 can contain hydrocarbons containing 2 to 3 carbon atoms derived from any source, e.g., a fossil-based source, a renewable source (bio-based renewable feedstock), or a circular source. For example, the C2 hydrocarbons and C3 hydrocarbons in feed stream 11 can be recovered or derived from natural gas liquids (NGLs), fossil fuels, bio-based feedstocks (e.g., vegetable oils, natural oils), a refinery C2-C3 hydrocarbon stream (e.g., a refinery stream containing 60 wt % to 80 wt % C2 compounds, a refinery stream containing 60 wt % to 80 wt % propylene, or both), or a combination thereof. In aspects, obtaining the C2 hydrocarbons and C3 hydrocarbons from the feed source can produce a residual C4+hydrocarbons. In these aspects, the C4+ hydrocarbons derived from the feed sources can be fed to the fluid catalytic cracking unit 20 via stream 21 (e.g., via a bottom stream of a depropanizer that is coupled to stream 21). The C2 and C3 hydrocarbons can be derived from any of the feed sources according to any technique known in the art with the aid of this disclosure.

The operating conditions for the steam cracking unit 10 can be any conditions (e.g., feed flow rate, temperature, fuel flow rate, fuel type) known in the art with the aid of this disclosure such that one or more olefins (e.g., ethylene, propylene, or both) are produced from the cracking reactions in the steam cracking unit 10.

The steam cracking unit 10 produces a cracked gas in stream 12. Stream 12 is coupled to the steam cracking unit 10 and to the separation system 100.

The process can include cracking C2 hydrocarbons and C3 hydrocarbons in the steam cracking unit 10 to form a cracked gas. The process can also include separating a feed source (e.g., NGLs, fossil fuels, bio-based feedstocks, or a combination thereof) into i) the C2 hydrocarbons and the C3 hydrocarbons, and ii) a C4+ hydrocarbon product. The process can further include feeding the C2 hydrocarbons and the C3 hydrocarbons to the steam cracking unit 10 and feeding C4+ hydrocarbons comprising the C4+ hydrocarbon product to the fluid catalytic cracking unit 20. Separating, or segregating, the feed source can be accomplished by any technique known in the art with the aid of this disclosure such as distillation with a depropanizer as described above.

### Fluid Catalytic Cracking Unit

The fluid catalytic cracking unit (FCC unit) 20 can include a mixing zone, a catalytic cracking zone, a separation zone, and a regeneration zone. These zones form a loop through which catalyst particles circulate through the FCC unit 20. The FCC unit 20 is operated under high severity conditions, which include temperatures greater than 500° C.

The mixing zone can include one or more vessels configured to receive the feed to the FCC unit 20 and catalyst from a catalyst hopper or directly from the regeneration zone. The feed to the FCC unit 20 is mixed with catalyst in the mixing zone. The mixture of the feed components and the catalyst is then passed from the mixing zone and then introduced to the catalytic cracking zone. The catalytic cracking zone can include one or more vessels or pipes connected to the outlet(s) of the vessel(s) in the mixing zone. In aspects, the vessel(s) of the catalytic cracking zone are downflow reactors, or alternatively, upflow reactors. In some embodiments, steam and/or hydrogen can be introduced to an upstream portion of the vessel(s) in the catalytic cracking zone. In the catalytic cracking zone, the hydrocarbons in the feed components react with the catalyst, creating the reaction products in the catalytic cracking zone that can include spent catalyst and cracking product.

The cracking product can include a light cracking product and a heavy cracking product. The light cracking product can include hydrogen, light paraffins having 1 to 4 carbon atoms, and light olefins having 2 to 4 carbon atoms. The heavy cracking product can include naphtha, light cycle oil, slurry oil, and coke. The light cracking product can include hydrocarbons such as methane, ethane, propane, ethylene, propylene, butanes, butenes (1-butene, cis-2-butene, trans-2-butene, isobutylene (2-methylpropene), or a combination thereof), or combinations thereof. In aspects, the particles of catalyst can be covered with a layer of the coke that is formed as one of the cracking products, rendering the particles of catalyst as deactivated or spent catalyst.

Following the catalytic cracking reaction in the catalytic cracking zone, the cracking product and spent catalyst can pass from the catalytic cracking zone to the separation zone, where the spent catalyst covered with coke is separated from the other cracking products. The separation zone can include one or more vessels having one or more inlets fluidly connected to the outlet(s) of the vessel(s) in the catalytic cracking zone. In one or more embodiments, the separation zone can include one or more gas solid separators, such as one or more cyclone separators. The spent catalyst can pass from the separation zone to a stripping zone, and the remaining cracking product can flow from the separation zone to downstream processing and/or to product storage.

The stripping zone can include one or more vessels having inlet(s) fluidly connected to spent catalyst outlet(s) of the separation zone and outlet(s) fluidly connected to the inlet(s) of the regeneration zone. In the stripping zone, steam or nitrogen can be introduced in countercurrent direction with respect to a direction of flow of the spent catalyst through the vessel(s) of the stripping zone. Stripping can remove any residual cracking products contained among the spent catalyst particles. The stripped product containing residual cracking product and steam can pass to one or more downstream unit operations or be combined with one or more other streams for further processing.

The stripped spent catalyst can flow to the regeneration zone. The regeneration zone can include one or more vessels. The regeneration zone can also include one or more standpipe that is fluidly connected to the stripped spent catalyst outlet of the stripping zone vessel(s) and to an inlet to the vessel(s) of the regeneration zone. The stripped spent catalyst can be introduced to a bottom end of the standpipe, along with a transport medium comprising air, oxygen, fuel gas, fuel oil, or any combinations thereof. The transport medium can convey the stripped spent catalyst upwards (or downwards, depending on the flow design) through the standpipe to the vessel(s) of the regeneration zone. The coke deposited on the stripped spent catalyst can combust in the presence of the transport medium in the regenerator as the spent catalyst moves through the regeneration zone. Combustion of the coke removes the coke and sulfur species from the surface of the catalyst, regenerating the catalyst particles to form regenerated catalyst. The regenerated catalyst can then flow from the regeneration zone to the mixing zone. The catalyst is then recirculated through the FCC unit in a loop comprising the mixing zone, the catalytic cracking zone, the stripping zone, and the regeneration zone according to the process described above.

Cracking can be performed in the presence of a cracking catalyst, which can include a zeolite catalyst, a modified zeolite catalyst, a silica-alumina catalyst, a modified silica-alumina catalyst, a molecular sieve catalyst, a metal oxide catalyst, or a combination thereof. Examples of a zeolite catalyst includes a Y zeolite catalyst, a pentasil zeolite (MFI) catalyst, a beta zeolite (BEA) catalyst, a ferrierite zeolite (FER) catalyst, a chabazite zeolite (CHA) catalyst, or combinations thereof. Examples of Y zeolite catalyst include an HY zeolite and an ultrastable Y zeolite catalyst. An example of a pentasil zeolite catalyst is ZSM-5 zeolite catalyst. An example of a beta zeolite catalyst additive is H-Beta zeolite catalyst. Examples of modified zeolite catalyst include a rare-earth Y zeolite catalyst and a rare-earth ultrastable Y zeolite catalyst. Examples of a silica alumina catalyst include a silica-alumina catalyst having a pore size that is greater than the pore size of a zeolite catalyst. Examples of a modified silica-alumina catalyst include a chlorided silica-alumina catalyst and a fluorided silica-alumina catalyst.

In aspects, the amount of a pentasil zeolite (MFI) catalyst, a beta zeolite (BEA) catalyst, a ferrierite zeolite (FER) catalyst, a chabazite zeolite (CHA) catalyst, or combinations thereof present in the cracking catalyst can range from 20 wt % to 80 wt %, alternatively from 30 wt % to 80 wt %, alternatively from 40 wt % to 80 wt % based on a total weight of the cracking catalyst. Use of these catalysts can be beneficial especially for operation of the FCC unit 20 under severe conditions.

The preparation of the modified zeolite catalyst and/or the modified silica-alumina catalyst can be according to any technique known in the art with the aid of this disclosure.

For example, a fluorided silica-alumina catalyst can be formed by contacting silica-alumina particles with a fluoriding agent. The fluoride ion of the fluoriding agent can be added to the oxide by forming a slurry of the oxide in a suitable solvent such as alcohol or water. Examples of suitable fluoriding agents include, but are not limited to, hydrofluoric acid (HF), ammonium fluoride (NH₄F), ammonium bifluoride (NH₄HF₂), ammonium tetrafluoroborate (NH₄BF₄), ammonium silicofluoride (hexafluorosilicate) ((NH₄)₂SiF₆), ammonium hexafluorophosphate (NH₄PF₆), hexafluorotitanic acid (H₂TiF₆), ammonium hexafluorotitanic acid ((NH₄)₂TiF₆), hexafluorozirconic acid (H₂ZrF₆), AlF₃, NH₄AlF₄, or combinations thereof. The fluoriding agent can alternatively or additionally be a volatile organic fluoriding agent such as a freon, perfluorohexane, perfluorobenzene, fluoromethane, trifluoroethanol, or combinations thereof, which can be useful for fluoriding the silica-alumina particles during calcining. One method of contacting silica-alumina particles with the fluoriding agent is to vaporize the fluoriding agent into a gas stream and use the vaporized fluoriding agent to fluidize the silica-coated alumina during calcination.

In another example, a chlorided silica-alumina catalyst can be formed by contacting silica-alumina particles with a chloriding agent. The chloride ion can be added to the oxide by forming a slurry of the oxide in a suitable solvent. The silica-alumina particles can be treated with the chloriding agent while calcining the silica-alumina particles. Any chloriding agent capable of serving as a source of chloride and thoroughly contacting the oxide during the calcining step can be used, such as SiCl₄, SiMe₂Cl₂, TiCl₄, BCl₃, or combinations thereof. Volatile organic chloriding agents can be used. Examples of suitable volatile organic chloriding agents include, but are not limited to, certain freons, perchlorobenzene, chloromethane, dichloromethane, chloroform, carbon tetrachloride, trichloroethanol, or combinations thereof. Gaseous hydrogen chloride or chlorine gas can also be used with the solid oxide during calcining. One method of contacting silica-alumina with the chloriding agent is to vaporize a chloriding agent into a gas stream and using the gas stream to fluidize the solid oxide during calcination.

The amount of fluoride or chloride ion present before calcining the silica-alumina generally is from 1 wt % to 50 wt %; alternatively, from 1 wt % to 25 wt %; alternatively, from 2 wt % to 15 wt %; alternatively, from 3 wt % to 12 wt %; alternatively, from 5 wt % to 10 wt %, based on the weight of the silica-alumina. Once chlorided or fluorided, the halogenated silica-alumina particles can be dried by any suitable method.

In aspects, the silica-alumina particles can be calcined prior to use as FCC catalyst. In some aspects, the silica-alumina particles can be calcined prior to being chlorided or fluorided.

In aspects, the process includes cracking, in the FCC unit 20, a feed containing C4+ hydrocarbons into a fluid catalytic cracking (FCC) product. In aspects, the C4+ hydrocarbons include butanes, butenes, naphtha, a pyrolysis oil, a used lubricating oil, a bio-based feedstock (e.g., vegetable oil), a renewable feedstock (e.g., bio-based renewable feedstock such as oils derived from algae, plants, or agricultural waste), a non-renewable feedstock (e.g., crude oil, NGLs), a circular feedstock (e.g., a circular pyrolysis oil derived from waste plastics), or a combination thereof. The feed is received via stream 21, stream 23, or both stream 21 and stream 23.

Stream 21 can contain butanes, butenes, naphtha, or both. In aspects, the butanes, butenes, naphtha, or both are the C4+ hydrocarbon product that is recovered or derived from the same feed source from which the C2 hydrocarbons and C3 hydrocarbons are recovered or derived. In some aspects, stream 21 can be coupled to the same equipment from which the C2 and C3 hydrocarbons are derived for the steam cracking unit 10, where stream 21 feeds the residual C4+ hydrocarbons (e.g., butanes, butenes, naphtha, or a combination thereof) recovered or derived from the feed source to the FCC unit 20.

Stream 23 can contain a pyrolysis oil, a used lubricating oil, a bio-based feedstock, a renewable feedstock, a non-renewable feedstock, a circular feedstock, or a combination thereof. In some aspects, stream 21 and stream 23 can be combined into a single feed stream that connects to the FCC unit 20. In aspects, only the C4+ hydrocarbons of stream 21, stream 23, or both stream 21 and stream 23 (alternatively, as the same stream) are fed to the FCC unit 20.

In aspects, stream 23 can be the product of a cleanup unit that produces stream 23. In the cleanup unit, nitrogen can be removed from the pyrolysis oil, the used lubricating oil, the bio-based feedstock, the renewable feedstock, the fossil-based (i.e., non-renewable) feedstock, or a combination thereof, so that a concentration of nitrogen in the stream 23 is less than 3500 ppmw based on a total weight of stream 23. In some aspects, the concentration of nitrogen in stream 23 is in a range of from 100 ppmw to 900 ppmw based on a total weight of the stream 23. The cleanup unit can be configured to remove nitrogen, diolefins, halides, silicon, or combinations thereof.

In aspects where stream 23 contains a pyrolysis oil, the pyrolysis oil can be a waste plastic pyrolysis oil. In such aspects, stream 23 can be coupled to a pyrolysis unit 80 that thermally treats a polyolefin to produce a pyrolysis product comprising the pyrolysis oil that is fed to the FCC unit 20 via stream 23. The pyrolysis oil can be produced by thermally treating a polyolefin at pyrolysis conditions to form a pyrolysis product comprising the pyrolysis oil. “Pyrolysis” of polyolefins as used herein refers to the thermal treatment of polyolefins at a temperature of 200° C. or greater, such as in the range of 200°C. to 900° C. In some aspects, the waste plastic pyrolysis oil can have olefins present in a range of from 1 wt % to 99 wt % based on a total weight of the waste plastic pyrolysis oil; alternatively, the waste plastic pyrolysis oil contains no olefins.

Thermal treatment at a temperature in a range of from 200° C. to 600° C. can be referred to as thermal treatment under mild conditions. Mild conditions can include a temperature of less than 600° C.; alternatively, a temperature in a range of from 100° C. to less than 600° C.; alternatively, a temperature in a range of from 200° C. to less than 600° C.; alternatively, a temperature in a range of from 300° C. to less than 600° C.; alternatively, a temperature in a range of from 400° C. to less than 600° C.; alternatively, a temperature in a range of from 200° C. to less than 500° C.; alternatively, a temperature in a range of from 200° C. to less than 400° C.; alternatively, a temperature in a range of from 400° C. to less than 500° C.; alternatively, a temperature in a range of from 200° C. to less than 300° C.; alternatively, a temperature in a range of from 300° C. to less than 500° C.

Thermal treatment at a temperature of 600° C. or greater can also be referred to as thermal treatment under severe conditions. Severe conditions can include a temperature in a range of from 600° C. to 900° C.; alternatively, a temperature in a range of from 600° C. to 800° C.; alternatively, a temperature in a range of from 600° C. to 700° C.

The polyolefins used as feedstock for the pyrolysis unit 80 can include polyethylene, polypropylene, or a combination thereof. The polyolefins can be embodied as homopolymer, copolymer, or both homopolymer and copolymer. The polyethylene can be embodied as high density polyethylene (HDPE), low density polyethylene (LDPE), medium density polyethylene (MDPE), linear low density polyethylene (LLDPE), or combinations thereof. Generally, the polyolefins are solids prior to being introduced to the thermolysis reactor.

In aspects, the polyolefins fed to the pyrolysis unit 80 can be part of a feedstock in an amount that is greater than 70, 75, 80, 85, 90, or 95 wt % based on a total weight of the feedstock. In additional aspects, the feedstock can also include other types of plastic, such as polystyrene (PS), polyethylene terephthalate (PET), polyvinyl chloride (PVC), or combinations thereof, for example, in an amount that is less than 30, 25, 20, 15, 10, or 5 wt % based on the total weight of the feedstock. The polyolefins in the feedstock can be pre-processed, e.g., mechanically broken into pieces having a median size (as measured in a largest dimension of a piece of solid polyolefin) of equal to or less than 10, 9, 8, 7, 6, 5, 4, 3, 2, 1, or 0.5 cm. In some aspects, a solvent or liquid carrier can be added to pieces of solid polyolefins to form a solution or slurry containing the polyolefins, which can then be introduced to the inlet of pyrolysis unit 80 by a pump. Examples of suitable solvents can include (but are not limited to) water, crude oil, naphtha, kerosene, diesel, gas oil, vacuum gas oil, waste plastic pyrolysis oil, mineral oil, toluene, benzene, xylene, methylnaphthalene, cyclohexane, methylcyclohexane, or combinations thereof. In addition, the feedstock containing the polyolefin can be heated prior to introducing the feedstock to the pyrolysis unit 80.

The process can include thermally treating, in the pyrolysis unit 80, a polyolefin under pyrolysis conditions to produce a pyrolysis product containing a pyrolysis oil, and introducing the pyrolysis oil to the FCC unit 20.

### Separation System

The separation system 100 can include any combination of equipment that can separate the cracked gas and FCC product into the plurality of hydrocarbon products in streams 101, 102, 103, 104, 105, 106, 107, 108, and 109. The equipment can include a quench tower, a fractionation tower, a compressor(s), an olefin recovery train (e.g., a demethanizer, a deethanizer, a depropanizer, or combinations thereof), and equipment associated with those vessels such as reboilers, accumulators, condensers, valves, piping, control equipment, heat exchangers, and combinations thereof.

The process can include introducing the cracked gas from stream 12 and the FCC product from stream 22 to the separation system 100, and recovering the plurality of hydrocarbon products from the separation system 100 in streams 101, 102, 103, 104, 105, 106, 107, 108, and 109. The plurality of hydrocarbon products can include a methane product in stream 101, an ethane product in stream 102, a propane product in stream 103, an ethylene product in stream 104, a propylene product in stream 105, a heavy product in stream 106 (e.g., containing hydrocarbons having from 4 carbon atoms up to the cut made in the quench tower 120, for example, up to 10 carbon atoms), a fuel oil in stream 107, a light cycle oil in stream 108, and a slurry oil in stream 109. In aspects, circular ethylene is recovered in stream 104, and circular propylene is recovered in stream 105.

In aspects, stream 102 containing the ethane product can be coupled to the steam cracking unit 10. The process can include flowing or recycling the ethane product to the steam cracking unit 10. In aspects, stream 103 containing the propane product can be coupled to the steam cracking unit 10. The process can include flowing or recycling the propane product to the steam cracking unit 10. In aspects, stream 102 and stream 103 can be coupled to the steam cracking unit 10. The process can include flowing or recycling the ethane product and the propane product to the steam cracking unit 10. The ethane product in stream 102, the propane product in stream 103, or both can be used as fuel in the fuel fired furnace of the steam cracking unit 10. In these aspects where the ethane product, propane product, or both are recycled as fuel for the steam cracking unit 10, no C4 compound is contained in the streams (e.g., stream 102, stream 103, or both) that are fed to the steam cracking unit 10. In aspects, no C4 compound in recycled from the separation system 100 to the steam cracking unit 10.

### Ethylene Polymerization Reactor

Circular polyethylene can be produced in the ethylene polymerization reactor 30. In aspects, the ethylene polymerization reactor 30 is coupled to the separation system 100 via stream 104. The ethylene polymerization reactor 30 can be embodied as one or more loop slurry reactors, gas phase reactors (also known as fluidized bed reactors), stirred tank reactors, axial flow reactors, and horizontal gas phase reactors. In the ethylene polymerization reactor 30, a reaction medium containing or consisting of the circular ethylene received from stream 104 is contacted with a polymerization catalyst (e.g., a Phillips catalyst, a Ziegler catalyst, a Ziegler-Natta catalyst, or a metallocene catalyst) while the reaction medium is circulated, stirred, or fluidized in the ethylene polymerization reactor 30, forming a polymer product that can be withdrawn from the polymerization reactor 30 and subjected to various separations (e.g., flashline heating, flashing, degassing, and combinations thereof) to recover the solid polymer particles of circular polyethylene called “polymer fluff” in polyethylene stream 31. The circular polyethylene can be a homopolymer, copolymer, or a multimodal polymer comprising polyethylene. The operating conditions of the ethylene polymerization reactor 30 can be any conditions (e.g., monomer feed rate, temperature, pressure, catalyst feed rate, etc.) known in the art with the aid of this disclosure.

The process can include introducing the circular ethylene from stream 104 into the polymerization reactor 30 and polymerizing the circular ethylene to produce a polyolefin product containing circular polyethylene in stream 31.

### Ethylene Oligomerization Reactor

Additionally or alternatively, circular oligomers can be produced in the ethylene oligomerization reactor 40. The ethylene oligomerization reactor 40 can be coupled to the separation system 100 via stream 104. The ethylene oligomerization reactor 40 can be embodied as any reactor or vessel such as a flow reactor, a continuous reactor, a packed tube, or a stirred tank reactor, including more than one reactor in series or in parallel, and including any combination of reactor types and arrangements. The ethylene oligomerization reactor 40 can contact the circular ethylene received from stream 104 with an oligomerization catalyst (e.g., a Lewis acid such as BF₃, BCl₃, AlCl₃, AlBr₃, TiCl₃, TiBr₃, TiCl₄, TiBr₄, SnCl₄, GaCl₃, GaBr₃, FeCl₃, FeBr₃, synthetic or natural zeolites, acid clays, polymeric acidic resins, amorphous solid catalysts such as silica-alumina, heteropolyacids such as tungsten zirconates, tungsten molybdates, tungsten vanadates, phosphotungstates and molybdotungstovanadogermanates (e.g., WOx/ZrO₂, WOx/MoO₃), an alkylaluminum halide, an aluminum trihalide, or combinations thereof) to form an oligomer product containing circular oligomers that are recovered in stream 41. The operating conditions of the ethylene oligomerization reactor 40 can be any conditions (e.g., monomer feed rate, temperature, pressure, catalyst feed rate, etc.) known in the art with the aid of this disclosure.

The process can include introducing the circular ethylene to the oligomerization reactor 40, and oligomerizing the circular ethylene in the oligomerization reactor 40 to produce an oligomer product containing circular oligomers in stream 41.

### Polyalphaolefin Production System

Additionally or alternatively, circular polyalphaolefins can be produced in a system that includes an oligomerization reactor 50 followed by a hydrogenation reactor 60. The oligomerization reactor 50 can be coupled to the separation system 100 via stream 104, and the hydrogenation reactor 60 can be coupled to the oligomerization reactor 50. Circular ethylene received from stream 104 can be oligomerized in the oligomerization reactor 50 and then the circular oligomers can be hydrogenated in the hydrogenation reactor 60 to form a circular polyalphaolefin product.

In aspects, the oligomerization reactor 50 can be the same as oligomerization reactor 40. The ethylene oligomerization reactor 50 can be embodied any reactor or vessel such as a flow reactor, a continuous reactor, a packed tube, or a stirred tank reactor, including more than one reactor in series or in parallel, and including any combination of reactor types and arrangements. The ethylene oligomerization reactor 50 can contact the circular ethylene received from stream 104 with an oligomerization catalyst (e.g., a Lewis acid such as BF₃, BCl₃, AlCl₃, AlBr₃, TiCl₃, TiBr₃, TiCl₄, TiBr₄, SnCl₄, GaCl₃, GaBr₃, FeCl₃, FeBr₃, synthetic or natural zeolites, acid clays, polymeric acidic resins, amorphous solid catalysts such as silica-alumina, heteropolyacids such as tungsten zirconates, tungsten molybdates, tungsten vanadates, phosphotungstates and molybdotungstovanadogermanates (e.g., WOx/ZrO₂, WOx/MoO₃), an alkylaluminum halide, an aluminum trihalide, or combinations thereof) to form an oligomer product containing circular oligomers that are recovered in stream 51. The operating conditions of the ethylene oligomerization reactor 50 can be any conditions (e.g., monomer feed rate, temperature, pressure, catalyst feed rate, etc.) known in the art with the aid of this disclosure.

The circular oligomers in stream 51 can feed to the hydrogenation reactor 60. The hydrogenation reactor 60 can be embodied as a slurry reactor, a continuous stirred tank reactor, a fixed bed reactor or any combination thereof. Hydrogenation can be accomplished by any means known to those with ordinary skill in the art with the aid of this disclosure. In aspects, the oligomer product from the oligomerization reactor 50 in stream 50 can contain unreacted circular ethylene. All or a portion of the oligomer product can be separated from the unreacted circular ethylene. The separated circular oligomers can be fed via stream 51 to hydrogenate unsaturated double bonds in the hydrogenation reactor 60, producing a polyalphaolefin product comprising circular polyalphaolefins. Generally, hydrogenation can include contacting the circular oligomers and a hydrogenation catalyst to form the circular polyalphaolefin under hydrogenation conditions. In aspects, the hydrogenation catalyst can include a supported Group 7, 8, 9, and 10 metal. In some aspects, the hydrogenation catalyst can be selected from one or more of Ni, Pd, Pt, Co, Rh, Fe, Ru, Os, Cr, Mo, and W, supported on silica, alumina, clay, titania, zirconia, or a mixed metal oxide support. In other aspects, the hydrogenation catalyst can be nickel supported on kieselguhr, platinum or palladium supported on alumina, or cobalt-molybdenum supported on alumina; alternatively, nickel supported on kieselguhr; alternatively, platinum or palladium supported on alumina; or alternatively, cobalt-molybdenum supported on alumina. In yet other aspects, the hydrogenation catalyst can be one or more of the group consisting of nickel supported on kieselguhr, silica, alumina, clay or silica-alumina. The operating conditions of the hydrogenation reactor 60 can be any conditions (e.g., monomer feed rate, temperature, pressure, catalyst feed rate, etc.) known in the art with the aid of this disclosure.

The process can include introducing the circular ethylene to the oligomerization reactor 50, and oligomerizing the circular ethylene in the oligomerization reactor 50 to produce an oligomer product containing circular oligomers in stream 51. The hydrogenation reactor 60 is coupled to the oligomerization reactor 50 via stream 51. The process can include hydrogenating the circular oligomers to produce a polyalphaolefin product containing circular polyalphaolefins in stream 61.

### Propylene Polymerization Reactor

Additionally or alternatively, circular polypropylene can be produced in the propylene polymerization reactor 70. The propylene polymerization reactor 70 can be coupled to the separation system 100 via stream 105. The circular propylene in stream 105 can be used for the production of circular polypropylene in the propylene polymerization reactor 70. The propylene polymerization reactor 70 can be embodied as one or more loop slurry reactors, gas phase reactors (also known as fluidized bed reactors), stirred tank reactors, axial flow reactors, and horizontal gas phase reactors. In the propylene polymerization reactor 70, a reaction medium containing or consisting of the circular propylene received from stream 105 is contacted with a polymerization catalyst (e.g., a Phillips catalyst, a Ziegler catalyst, a Ziegler-Natta catalyst, or a metallocene catalyst) while the reaction medium is circulated, stirred, or fluidized in the propylene polymerization reactor 70, forming a polymer product that can be withdrawn from the polymerization reactor 70 and subjected to various separations (e.g., flashline heating, flashing, degassing, and combinations thereof) to recover the solid polymer particles of circular polypropylene called “polymer fluff” in polypropylene stream 71. The circular polypropylene can be a homopolymer, copolymer, or a multimodal polymer comprising polypropylene. The operating conditions of the propylene polymerization reactor 70 can be any conditions (e.g., monomer feed rate, temperature, pressure, catalyst feed rate, etc.) known in the art with the aid of this disclosure.

The process can include introducing circular propylene to the polymerization reactor 70 via stream 105, and polymerizing the circular propylene in the polymerization reactor 70 to produce a polyolefin product containing circular polypropylene in stream 71.

Other circular product production systems can utilize the ethylene of stream 104 and/or propylene in stream 105. For example, it is intended that the scope of this disclosure include a styrene reactor, where benzene from a benzene source and ethylene from stream 104 can be reacted to produce ethylbenzene. The ethylbenzene can then be dehydrogenated in a dehydrogenation reactor known in the art with the aid of this disclosure to form styrene. Styrene can then be polymerized in a styrene polymerization reactor to form polystyrene product.

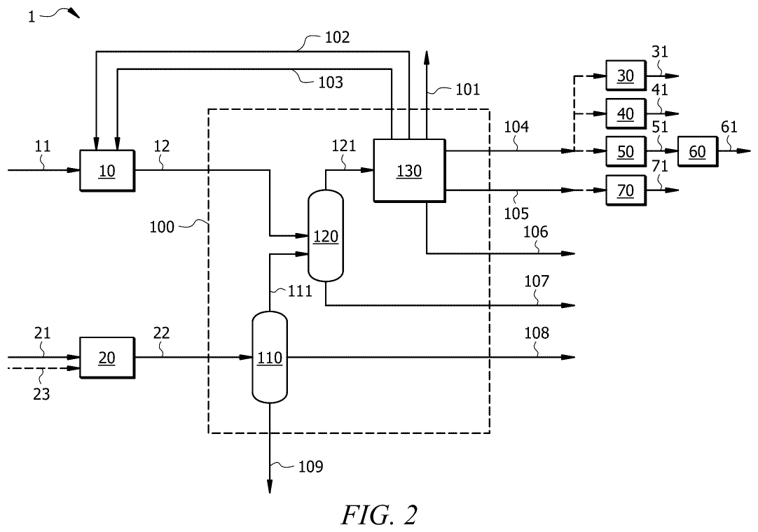

### FIG. 2

FIG. 2 is a schematic diagram of the system 1 of FIG. 1, having a first embodiment for the separation system 100. In FIG. 2, the separation system 100 includes a fractionation tower 110, a quench tower 120, and an olefin recovery train 130. In the embodiment of the separation system 100 in FIG. 2, the olefin recovery train 130 produces the methane product in stream 101, the ethane product in stream 102, the propane product in stream 103, the ethylene product in stream 104, and the propylene product in stream 105; the quench tower 120 produces the fuel oil in stream 107; and the fractionation tower 110 produces the light cycle oil in stream 108 and the slurry oil in stream 109.

The fractionation tower 110 can be embodied as one or more distillation columns having trays, packing, baffles, or any internal structuring known in the art with the aid of this disclosure for separation of the components of FCC product stream 22 into streams 111, 108, and 109. Stream 111 is an overhead product of the fractionation tower 110 that flows and connects with the quench tower 120 of the separation system 100. While stream 12 connects to the side of the quench tower 120 at a location above where the stream 111 connects to the quench tower 120, the relative spacing is illustrative only, and it is contemplated that stream 111 can connect to the side of the quench tower 120 above the location where stream 12 connects to the quench tower 120. In aspects, stream 108 is a side stream connected to a side of the fractionation tower 110, and stream 109 is a bottom stream connected to the bottom of the fractionation tower 110.

The quench tower 120 can be embodied as one or more distillation columns having trays, packing, baffles, or any internal structuring known in the art with the aid of this disclosure for separation of the components of cracked gas stream 12 into streams 121 and 107. The quench tower 120 separates the cracked gas received from stream 12 and the first overhead product received from stream 111 into a fuel oil in stream 107 and a second overhead product in stream 121. The cut made in the quench tower 120 between streams 121 and 107 can be a C10 hydrocarbon cut, so that C10 and smaller hydrocarbons flow in stream 121 to the olefin recovery train 130 and C11+ hydrocarbons having a boiling point in a range of the boiling point for C11 hydrocarbons to about 400° F. (204.4° C.) flow in the fuel oil of stream 107.

Stream 121 can contain hydrocarbons lighter than the cut taken in the quench tower 120, for example, C10 and lighter hydrocarbons. For example, the components in stream 121 can have a boiling point range of less than the boiling point of C11 hydrocarbons (e.g., about 385° F. (196.1° C.)). In aspects, stream 121 includes hydrogen.

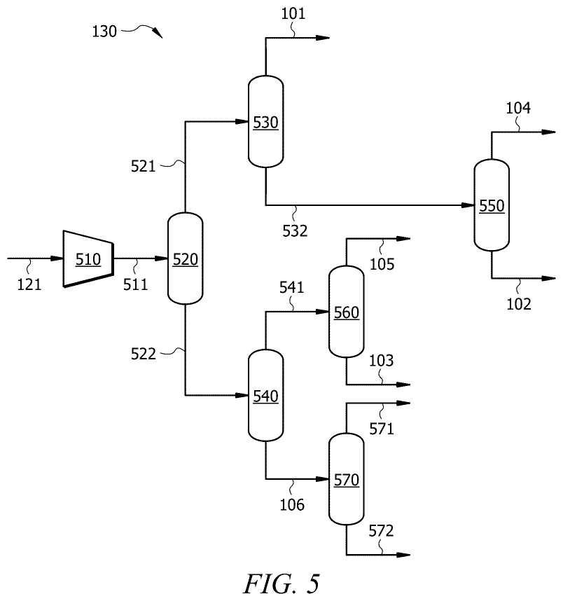

The olefin recovery train 130 has one or more distillation columns (e.g., one or more of a demethanizer, deethanizer, depropanizer, debutanizer, olefin/paraffin splitters, and associated equipment) and associated equipment that collectively separate the second overhead product in stream 121 into the plurality of hydrocarbon products in streams 101, 102, 103, 104, 105, and 106. An example of an olefin recovery train 130 is illustrated in FIG. 5 and described in more detail herein.

For the separation system 100 illustrated in FIG. 2, the process can include distilling the FCC product from stream 22 in the fractionation tower 110 to produce a first overhead product in stream 111, a light cycle oil in stream 108, and a slurry oil in stream 109. The process can also include distilling the cracked gas from stream 12 and the first overhead product received from stream 111 in the quench tower 120 to produce a fuel oil in stream 107 and a second overhead product in stream 121; and separating the second overhead product in stream 121 in the olefin recovery train 130 into the methane product in stream 101, the ethane product in stream 102, the propane product in stream 103, the ethylene product in stream 104, the propylene product in stream 105, and the heavy product in stream 106.

Stream 107 contains fuel oil having a boiling point range of from the boiling point of C11 hydrocarbons (e.g., about 385° F. (196.1° C.)) to about 400° F. (204.4° C.). Stream 108 contains a light cycle oil having a boiling point range of from about 400° F. (204.4° C.) to about 650° F. (343.3° C.). Stream 109 contains a slurry oil having a boiling point range of equal to and greater than about 650° F. (343.3° C.).

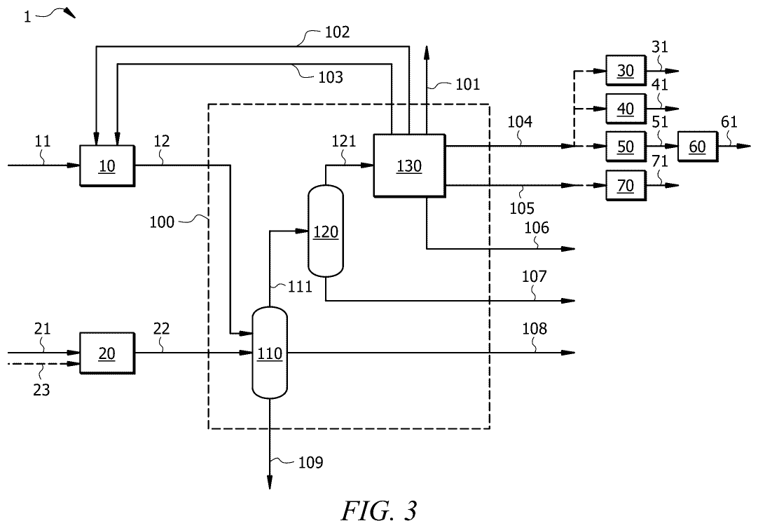

### FIG. 3

FIG. 3 is a schematic diagram of the system 1 of FIG. 1, having a second embodiment for the separation system 100. In FIG. 3, the separation system 100 includes a fractionation tower 110, a quench tower 120, and an olefin recovery train 130. In the embodiment of the separation system 100 in FIG. 3, the olefin recovery train 130 produces the methane product in stream 101, the ethane product in stream 102, and the propane product in stream 103, the ethylene product in stream 104, and the propylene product in stream 105; the quench tower 120 produces the fuel oil in stream 107; and the fractionation tower 110 produces the light cycle oil in stream 108 and the slurry oil in stream 109.

The fractionation tower 110 can be embodied as one or more distillation columns having trays, packing, baffles, or any internal structuring known in the art with the aid of this disclosure for separation of the components of cracked gas in stream 12 and the FCC product in stream 22 into streams 111, 108, and 109. While stream 12 connects to the side of the fractionation tower 110 at a location above where the stream 22 connects to the fractionation tower 110, the relative spacing is illustrative only, and it is contemplated that stream 22 can connect to the side of the fractionation tower 110 above the location where stream 11 connects to the fractionation tower 110. Stream 111 is an overhead product of the fractionation tower 110 that flows and connects with the quench tower 120 of the separation system 100. In aspects, stream 108 is a side stream connected to a side of the fractionation tower 110, and stream 109 is a bottom stream connected to the bottom of the fractionation tower 110.

The quench tower 120 can be embodied as one or more distillation columns having trays, packing, baffles, or any internal structuring known in the art with the aid of this disclosure for separation of the components of the overhead product in stream 111 into streams 121 and 107. The quench tower 120 separates the overhead product received from stream 111 into a fuel oil in stream 107 and a second overhead product in stream 121. The cut made in the quench tower 120 between streams 121 and 107 can be a C10 hydrocarbon cut, so that C10 and smaller hydrocarbons flow in stream 121 to the olefin recovery train 130 and C11+ hydrocarbons having a boiling point in a range of from the boiling point of C11 hydrocarbons to about 400° F. (204.4° C.) flow in the fuel oil of stream 107.

Stream 121 can contain hydrocarbons lighter than the cut taken in the quench tower 120, for example, C10 and lighter hydrocarbons. For example, the components in stream 121 can have a boiling point range of less than the boiling point of C11 hydrocarbons (e.g., about 385° F. (196.1° C.)). In aspects, stream 121 includes hydrogen.

The olefin recovery train 130 has one or more distillation columns (e.g., one or more of a demethanizer, deethanizer, depropanizer, debutanizer, olefin/paraffin splitters, and associated equipment) and associated equipment that collectively separate the second overhead product in stream 121 into the plurality of hydrocarbon products in streams 101, 102, 103, 104, 105, and 106. An example of an olefin recovery train 130 is illustrated in FIG. 5 and described in more detail herein.

For the separation system 100 illustrated in FIG. 3, the process can include distilling the cracked gas of stream 12 and the FCC product of stream 22 in a fractionation tower 110 to produce a first overhead product in stream 111, a light cycle oil in stream 108, and a slurry oil in stream 109; distilling the first overhead product from stream 111 in a quench tower 120 to produce a second overhead product in stream 121 and a fuel oil in stream 107; and separating the second overhead product in stream 121 into the methane product in stream 101, the ethane product in stream 102, the propane product in stream 103, the ethylene product in stream 104, the propylene product in stream 105, and the heavy product in stream 106.

Stream 107 contains fuel oil having a boiling point range of from the boiling point of C11 hydrocarbons (e.g., about 385° F. (196.1° C.)) to about 400° F. (204.4° C.). Stream 108 contains a light cycle oil having a boiling point range of from about 400° F. (204.4° C.) to about 650° F. (343.3° C.). Stream 109 contains a slurry oil having a boiling point range of equal to and greater than about 650° F. (343.3° C.).

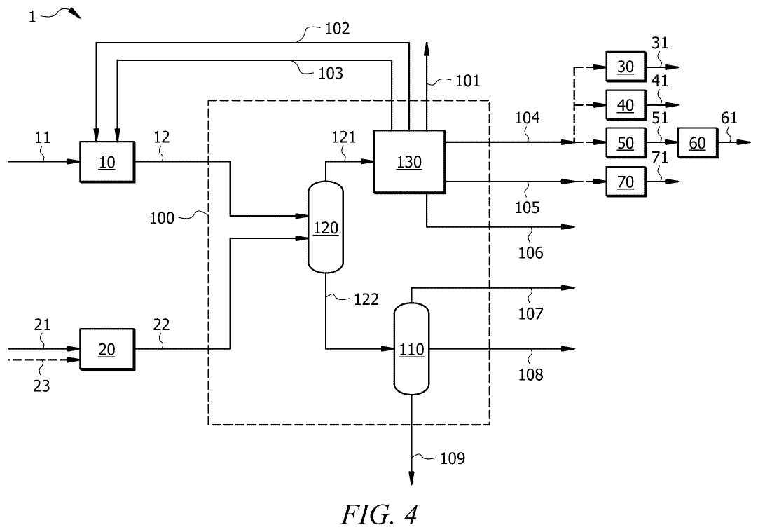

### FIG. 4

FIG. 4 is a schematic diagram of the system 1 of FIG. 1, having a third embodiment for the separation system 100. In FIG. 4, the separation system 100 includes a fractionation tower 110, a quench tower 120, and an olefin recovery train 130. In the embodiment of the separation system 100 in FIG. 4, the olefin recovery train 130 produces the methane product in stream 101, the ethane product in stream 102, and the propane product in stream 103, the ethylene product in stream 104, and the propylene product in stream 105; and the fractionation tower 110 produces the fuel oil in stream 107, the light cycle oil in stream 108, and the slurry oil in stream 109.

The quench tower 120 can be embodied as one or more distillation columns having trays, packing, baffles, or any internal structuring known in the art with the aid of this disclosure for separation of the components received from crack gas in stream 12 and FCC product in stream 22 into an overhead product in stream 121 and a bottom product in stream 122. The quench tower 120 separates the cracked gas received from stream 12 and the FCC product received from stream 22 into overhead product in stream 121 and bottom product in stream 122. While stream 12 connects to the side of the quench tower 120 at a location above where the stream 22 connects to the quench tower 120, the relative spacing is illustrative only, and it is contemplated that stream 22 can connect to the side of the quench tower 120 above the location where stream 11 connects to quench tower 120. The cut made in the quench tower 120 between streams 121 and 122 can be a C10 hydrocarbon cut, so that C10 and smaller hydrocarbons flow in stream 121 to the olefin recovery train 130 and C11+ hydrocarbons flow in the bottom product of stream 122 to the fractionation tower 110.

The fractionation tower 110 can be embodied as one or more distillation columns having trays, packing, baffles, or any internal structuring known in the art with the aid of this disclosure for separation of the components of bottom product of the quench tower 120 in stream 122 into streams 107, 108, and 109. Stream 122 is a bottom product of the quench tower 120 that flows and connects with the fractionation tower 110 of the separation system 100. In aspects, stream 107 is an overhead stream connected to a top of the fractionation tower 110, stream 108 is a side stream connected to a side of the fractionation tower 110, and stream 109 is a bottom stream connected to the bottom of the fractionation tower 110. Stream 107 contains fuel oil having a boiling point range of from the boiling point of C11 hydrocarbons (e.g., about 385° F. (196.1° C.)) to about 400° F. (204.4° C.). Stream 108 contains a light cycle oil having a boiling point range of from about 400° F. (204.4° C.) to about 650° F. (343.3° C.). Stream 109 contains a slurry oil having a boiling point range of equal to and greater than about 650° F. (343.3° C.).

Stream 121 can contain hydrocarbons lighter than the cut taken in the quench tower 120, for example, C10 and lighter hydrocarbons. For example, the components in stream 121 can have a boiling point range of less than the boiling point of C11 hydrocarbons (e.g., about 385° F. (196.1° C.)). In aspects, stream 121 includes hydrogen.

The olefin recovery train 130 has one or more distillation columns (e.g., one or more of a demethanizer, deethanizer, depropanizer, debutanizer, olefin/paraffin splitters, and associated equipment) and associated equipment that collectively separate the second overhead product in stream 121 into the plurality of hydrocarbon products in streams 101, 102, 103, 104, 105, and 106. An example of an olefin recovery train 130 is illustrated in FIG. 5 and described in more detail herein.

For the separation system 100 illustrated in FIG. 4, the process can include distilling the cracked gas of stream 12 and the FCC product of stream 22 in a quench tower 120 to produce a first overhead product in stream 121 and a bottom product in stream 122; distilling the bottom product from stream 122 in a fractionation tower 110 to produce a fuel oil in stream 107, a light cycle oil in stream 108, and a slurry oil in stream 109; and separating the overhead product in stream 121 into the methane product in stream 101, the ethane product in stream 102, the propane product in stream 103, the ethylene product in stream 104, the propylene product in stream 105, and the heavy product in stream 106.

### FIG. 5

FIG. 5 is a schematic diagram of an embodiment for the olefin recovery train 130 that can be utilized in any embodiment of the separation system 100 of FIGS. 1, 2, 3, and 4 disclosed herein.

The olefin recovery train 130 includes a compressor 510, a deethanizer 520, a demethanizer 530, a depropanizer 540, an ethane/ethylene splitter 550, a propane/propylene splitter 560, and a debutanizer 570. The order of the equipment in FIG. 5 is exemplary with regard to the direction of flow of products from left to right in FIG. 5, and other configurations can be utilized in which the order for the deethanizer 520, the demethanizer 530, the depropanizer 540, the ethane/ethylene splitter 550, the propane/propylene splitter 560, and the debutanizer 570 is different than shown in FIG. 5.

The overhead product in stream 121 from the quench tower 120 is received to a compressor 510. Stream 121 can contain hydrogen, methane, ethane, ethylene, propane, propylene, and C4 to C10 hydrocarbons. C10 hydrocarbons are exemplary because of the “cut” described herein for the quench tower 120. In aspects where the cut in the quench tower 120 is for hydrocarbon having a different carbon number, then the contents of stream 121 would contain corresponding components of C4 to CX (X being whatever numeral the carbon number is for the cut in the quench tower 120). The compressor 510 can be embodied as one or more gas compressors that compress the components of stream 121 to a pressure suitable for the olefin recovery in the olefin recovery train 130. Compression in compressor 510 produces compressed gas in stream 511. The compressed gas is fed to the deethanizer 520.

The deethanizer 520 can be embodied as one or more distillation columns having trays, packing, baffles, or any internal structuring known in the art with the aid of this disclosure for separation of the components of compressed gas in stream 511 into a deethanizer overhead comprising hydrogen, methane, ethane, and ethylene in stream 521 and a deethanizer bottoms comprising C3+ hydrocarbons in stream 522. The C3+ hydrocarbons can include propane, propylene, butanes, butenes, and C5+ hydrocarbons (those hydrocarbons C5 and larger that are not part of the fuel oil recovered in stream 107 from the separation system 100).

Stream 521 connects to the demethanizer 530. The demethanizer 530 can be embodied as one or more distillation columns having trays, packing, baffles, or any internal structuring known in the art with the aid of this disclosure for separation of the components of stream 521 into a demethanizer overhead comprising hydrogen and methane in stream 101 and a demethanizer bottoms comprising ethane and ethylene in stream 532.

Stream 532 can connect to an ethane/ethylene splitter 550. The ethane/ethylene splitter 550 can be embodied as one or more vessels configured to recover ethylene in the ethylene product in stream 104 and ethane in the ethane product in stream 102. Stream 104 is the stream that connects to one or more of the ethylene polymerization reactor 30, the ethylene oligomerization reactor 40, and the ethylene oligomerization reactor 50 of the polyalphaolefin system illustrated in FIGS. 1, 2, 3, and 4. Stream 102 is the stream that can connect to the steam cracking unit 10 in FIGS. 1, 2, 3, and 4.

Stream 522 connects to the depropanizer 540. The depropanizer 540 can be embodied as one or more distillation columns having trays, packing, baffles, or any internal structuring known in the art with the aid of this disclosure for separation of the components of stream 522 into a depropanizer overhead comprising propane and propylene in stream 541 and a depropanizer bottoms comprising C4+ hydrocarbons in the heavy product stream 106. The C4+ hydrocarbons include butanes, butenes, and C5+ hydrocarbons (those hydrocarbons C5 and larger that are not part of the fuel oil recovered in stream 107 from the separation system 100).

Stream 541 can connect to the propane/propylene splitter 560. The propane/propylene splitter 560 can be embodied as one or more vessels configured to recover propylene in the propylene product in stream 105 and propane in the propane product in stream 103. Stream 105 is the stream that connects to the propylene polymerization reactor 70 illustrated in FIGS. 1, 2, 3, and 4. Stream 103 is the stream that can connect to the steam cracking unit 10 in FIGS. 1, 2, 3, and 4.

The heavy product stream 106 connects to the debutanizer 570. The debutanizer 570 can be embodied as one or more distillation columns having trays, packing, baffles, or any internal structuring known in the art with the aid of this disclosure for separation of the components of stream 106 into a debutanizer overhead comprising C4 hydrocarbons in stream 571 and a debutanizer bottoms comprising C5+ hydrocarbons in stream 572. Stream 571 can contain C4 hydrocarbons, in some cases referred to as a butadiene feedstock (BDFS) that can be sold and/or used for the production of synthetic rubber, latex, nylon, and various butadienestyrene resins. The C5+ hydrocarbons are those hydrocarbons C5 and larger that are not part of the fuel oil recovered in stream 107 from the separation system 100.

The operating conditions for the compressor 510, the deethanizer 520, the demethanizer 530, the depropanizer 540, the ethane/ethylene splitter 550, the propane/propylene splitter 560, and the debutanizer 570 can be those known in the art for such separations.

### ADDITIONAL DESCRIPTION

Aspect 1. A process comprising: cracking C2 hydrocarbons and C3 hydrocarbons in a steam cracking unit to form a cracked gas; cracking C4+ hydrocarbons in a fluid catalytic cracking unit to form a fluid catalytic cracking (FCC) product; introducing the cracked gas and the FCC product to a separation system; and recovering a plurality of hydrocarbon products from the separation system, wherein the plurality of hydrocarbon products comprises one or more circular olefin.
Aspect 2. The process of Aspect 1, wherein recovering a plurality of hydrocarbon products from the separation system comprises: distilling the FCC product in a fractionation tower of the separation system to produce a first overhead product, a light cycle oil, and a slurry oil; distilling the cracked gas and the first overhead product in a quench tower of the separation system to produce a fuel oil and a second overhead product; and separating the second overhead product into the plurality of hydrocarbon products.
Aspect 3. The process of Aspect 1, wherein recovering a plurality of hydrocarbon products from the separation system comprises: distilling the cracked gas and the FCC product in a fractionation tower of the separation system to produce a first overhead product, a light cycle oil, and a slurry oil; distilling the first overhead product in a quench tower of the separation system to produce a second overhead product and a fuel oil; and separating the second overhead product into the plurality of hydrocarbon products.
Aspect 4. The process of Aspect 1, wherein recovering a plurality of hydrocarbon products from the separation system comprises: distilling the cracked gas and the FCC product in a quench tower of the separation system to produce an overhead product and a bottom product; distilling the bottom product in a fractionation tower of the separation system to produce a fuel oil, a light cycle oil, and a slurry oil; and separating the overhead product into the plurality of hydrocarbon products.
Aspect 5. The process of any one of Aspects 1 to 4, wherein the plurality of hydrocarbon products comprises a methane product, an ethane product, a propane product, an ethylene product, a propylene product, or combinations thereof.
Aspect 6. The process of Aspect 5, further comprising: flowing the ethane product and the propane product to the steam cracking unit.
Aspect 7. The process of Aspect 5 or 6, further comprising: introducing the ethylene product to a polymerization reactor or to an oligomerization reactor; and polymerizing the ethylene product in the polymerization reactor to produce a circular polyolefin product or oligomerizing the ethylene product in the oligomerization reactor to produce a circular oligomer product.
Aspect 8. The process of Aspect 7, wherein the ethylene product is oligomerized in the oligomerization reactor to form the circular oligomer product, the process further comprising: hydrogenating the circular oligomer product to form a circular polyalphaolefin product.
Aspect 9. The process of Aspect 5 or 6, further comprising: introducing the propylene product to a polymerization reactor; and polymerizing the propylene product in the polymerization reactor to produce a circular polyolefin product.
Aspect 10A. The process of any one of Aspects 1 to 9, wherein the C4+ hydrocarbons comprise butanes, butenes, naphtha, a pyrolysis oil, a used lubricating oil, a bio-based feedstock, a renewable feedstock, a non-renewable feedstock, a circular feedstock, or a combination thereof.
Aspect 10B. The process of any one of Aspects 1 to 10A, wherein only the C2 hydrocarbons and the C3 hydrocarbons are fed to the steam cracking unit.
Aspect 10C. The process of any one of Aspects 1 to 10B, wherein only the C4+ hydrocarbons are fed to the FCC unit.
Aspect 10D. The process of any one of Aspects 1 to 10C, wherein no naphtha is fed to the steam cracking unit.
Aspect 10E. The process of any one of Aspects 1 to 10D, further comprising: separating a feed source into i) the C2 hydrocarbons and the C3 hydrocarbons, and ii) a C4+ hydrocarbon product; feeding the C2 hydrocarbons and the C3 hydrocarbons to the steam cracking unit; and feeding the C4+ hydrocarbons comprising the C4+ hydrocarbon product to the fluid catalytic cracking unit.
Aspect 11. The process of any one of Aspects 1 to 10, wherein the C4+ hydrocarbons comprise a pyrolysis oil, the process further comprising: thermally treating a polyolefin to form a pyrolysis product comprising the pyrolysis oil; and introducing the pyrolysis oil to the fluid catalytic cracking unit.
Aspect 12. The process of any one of Aspects 1 to 11, wherein cracking C4+ hydrocarbons is performed under mild conditions or severe conditions.
Aspect 13. A circular olefin system comprising: a steam cracking unit that cracks C2 hydrocarbons and C3 hydrocarbons into a cracked gas; a fluid catalytic cracking unit that cracks C4+ hydrocarbons into a fluid catalytic cracking (FCC) product, wherein the C4+ hydrocarbons comprise butanes, butenes, naphtha, a pyrolysis oil, a used lubricating oil, a bio-based feedstock, a renewable feedstock, a non-renewable feedstock, a circular feedstock, or a combination thereof; and a separation system coupled to the steam cracking unit and to the fluid catalytic cracking unit, wherein the separation system receives the cracked gas and the FCC product, wherein the separation system produces a plurality of hydrocarbon products comprising one or more circular olefin.
Aspect 14. The circular olefin system of Aspect 13, further comprising: a polymerization reactor coupled to the separation system, wherein the polymerization reactor polymerizes the one or more circular olefin to produce a circular polyolefin product.
Aspect 15. The circular olefin system of Aspect 13 or 14, further comprising: an oligomerization reactor coupled to the separation system, wherein the oligomerization reactor oligomerizes the one or more circular olefin to produce a circular oligomer product.
Aspect 16. The circular olefin system of Aspect 15, further comprising: a hydrogenation reactor coupled to the oligomerization reactor, wherein the hydrogenation reactor hydrogenates the circular oligomer product to produce a circular polyalphaolefin product.
Aspect 17. The circular olefin system of any one of Aspects 13 to 16, wherein the separation system comprises: a fractionation tower that separates the FCC product into a first overhead product, a light cycle oil, and a slurry oil; a quench tower that separates the cracked gas and the first overhead product into a fuel oil and a second overhead product; and an olefin recovery train that separates the second overhead product into the plurality of hydrocarbon products.
Aspect 18. The circular olefin system of any one of Aspects 13 to 16, wherein the separation system comprises: a fractionation tower that separates the cracked gas and the FCC product into a first overhead product, a light cycle oil, and a slurry oil; a quench tower that separates the first overhead product into a second overhead product and a fuel oil; and an olefin recovery train that separates the second overhead product into the plurality of hydrocarbon products.
Aspect 19. The circular olefin system of any one of Aspects 13 to 16, wherein the separation system comprises: a quench tower that separates the cracked gas and the FCC product into an overhead product and a bottom product; a fractionation tower that separates the bottom product into a fuel oil, a light cycle oil, and a slurry oil; and an olefin recovery train that separates the overhead product into the plurality of hydrocarbon products.
Aspect 20. The circular olefin system of any one of Aspects 13 to 19, further comprising: a pyrolysis unit that thermally treats a polyolefin to produce a pyrolysis product comprising the pyrolysis oil, wherein the pyrolysis unit is coupled to the fluid catalytic cracking unit.
Aspect 21. The circular olefin system of any one of Aspects 13 to 20, wherein the steam cracking unit does not crack naphtha hydrocarbons.
Aspect 22. The circular olefin system of any one of aspects 13 to 20, further comprising a separator that separates a feed source into i) the C2 hydrocarbons and the C3 hydrocarbons, and ii) a C4+ hydrocarbon product, wherein the steam crack unit receives the C2 hydrocarbons and the C3 hydrocarbons, wherein the fluid catalytic cracking unit receives the C4+ hydrocarbons comprising the C4+ hydrocarbon product.
Aspect 23. A process for producing a circular olefin, comprising thermally treating a polyolefin to form a pyrolysis product comprising a pyrolysis oil; cracking C2 hydrocarbons and C3 hydrocarbons in a steam cracking unit to form a cracked gas; cracking the pyrolysis oil in a fluid catalytic cracking unit to form a fluid catalytic cracking (FCC) product; introducing the cracked gas and the FCC product to a separation system; recovering an olefin from the separation system; and introducing the olefin to a polymerization reactor or to an oligomerization reactor to produce another polyolefin.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods, and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A process comprising:
cracking C2 hydrocarbons and C3 hydrocarbons in a steam cracking unit to form a cracked gas;
cracking C4+ hydrocarbons in a fluid catalytic cracking unit to form a fluid catalytic cracking (FCC) product, wherein the C4+ hydrocarbons comprise butanes, butenes, naphtha, a pyrolysis oil, a used lubricating oil, a bio-based feedstock, a renewable feedstock, a non-renewable feedstock, a circular feedstock, or a combination thereof;
introducing the cracked gas and the FCC product to a separation system; and
recovering a plurality of hydrocarbon products from the separation system, wherein the plurality of hydrocarbon products comprises one or more circular olefin.

2. The process of claim 1, wherein recovering the plurality of hydrocarbon products from the separation system comprises:
distilling the FCC product in a fractionation tower of the separation system to produce a first overhead product, a light cycle oil, and a slurry oil;
distilling the cracked gas and the first overhead product in a quench tower of the separation system to produce a fuel oil and a second overhead product; and
separating the second overhead product into the plurality of hydrocarbon products.

3. The process of claim 1, wherein recovering the plurality of hydrocarbon products from the separation system comprises:
distilling the cracked gas and the FCC product in a fractionation tower of the separation system to produce a first overhead product, a light cycle oil, and a slurry oil;
distilling the first overhead product in a quench tower of the separation system to produce a second overhead product and a fuel oil; and
separating the second overhead product into the plurality of hydrocarbon products.

4. The process of claim 1, wherein recovering a plurality of hydrocarbon products from the separation system comprises:
distilling the cracked gas and the FCC product in a quench tower of the separation system to produce an overhead product and a bottom product;
distilling the bottom product in a fractionation tower of the separation system to produce a fuel oil, a light cycle oil, and a slurry oil; and
separating the overhead product into the plurality of hydrocarbon products.

5. The process of claim 1, wherein the plurality of hydrocarbon products comprises a methane product, an ethane product, a propane product, an ethylene product, a propylene product, or combinations thereof.

6. The process of claim 5, further comprising:
flowing the ethane product and the propane product to the steam cracking unit.

7. The process of claim 5, further comprising:
introducing the ethylene product to a polymerization reactor or to an oligomerization reactor; and
polymerizing the ethylene product in the polymerization reactor to produce a circular polyolefin product or oligomerizing the ethylene product in the oligomerization reactor to produce a circular oligomer product.

8. The process of claim 7, wherein the ethylene product is oligomerized in the oligomerization reactor to form the circular oligomer product, the process further comprising:
hydrogenating the circular oligomer product to form a circular polyalphaolefin product.

9. The process of claim 5, further comprising:
introducing the propylene product to a polymerization reactor; and
polymerizing the propylene product in the polymerization reactor to produce a circular polyolefin product.

10. The process of claim 1, further comprising:
separating a feed source into i) the C2 hydrocarbons and the C3 hydrocarbons, and ii) a C4+ hydrocarbon product;
feeding the C2 hydrocarbons and the C3 hydrocarbons to the steam cracking unit; and
feeding the C4+ hydrocarbons comprising the C4+ hydrocarbon product to the fluid catalytic cracking unit.

11. The process of claim 1, wherein no naphtha is fed to the steam cracking unit.

12. The process of claim 1, wherein the C4+ hydrocarbons comprise a pyrolysis oil, the process further comprising:
thermally treating a polyolefin to form a pyrolysis product comprising the pyrolysis oil; and
introducing the pyrolysis oil to the fluid catalytic cracking unit.

13. A circular olefin system comprising:
a steam cracking unit that cracks C2 hydrocarbons and C3 hydrocarbons into a cracked gas;
a fluid catalytic cracking unit that cracks C4+ hydrocarbons into a fluid catalytic cracking (FCC) product, wherein the C4+ hydrocarbons comprise butanes, butenes, naphtha, a pyrolysis oil, a used lubricating oil, a bio-based feedstock, a renewable feedstock, a non-renewable feedstock, a circular feedstock, or a combination thereof; and
a separation system coupled to the steam cracking unit and to the fluid catalytic cracking unit, wherein the separation system receives the cracked gas and the FCC product, wherein the separation system produces a plurality of hydrocarbon products comprising one or more circular olefin.

14. The circular olefin system of claim 13, further comprising:
a polymerization reactor coupled to the separation system, wherein the polymerization reactor polymerizes the one or more circular olefin to produce a circular polyolefin product.

15. The circular olefin system of claim 13, further comprising:
an oligomerization reactor coupled to the separation system, wherein the oligomerization reactor oligomerizes the one or more circular olefin to produce a circular oligomer product; and
optionally, a hydrogenation reactor coupled to the oligomerization reactor, wherein the hydrogenation reactor hydrogenates the circular oligomer product to produce a circular polyalphaolefin product.

16. The circular olefin system of claim 13, wherein the separation system comprises:
a fractionation tower that separates the FCC product into a first overhead product, a light cycle oil, and a slurry oil;
a quench tower that separates the cracked gas and the first overhead product into a fuel oil and a second overhead product; and
an olefin recovery train that separates the second overhead product into the plurality of hydrocarbon products.

17. The circular olefin system of claim 13, wherein the separation system comprises:
a fractionation tower that separates the cracked gas and the FCC product into a first overhead product, a light cycle oil, and a slurry oil;
a quench tower that separates the first overhead product into a second overhead product and a fuel oil; and
an olefin recovery train that separates the second overhead product into the plurality of hydrocarbon products.

18. The circular olefin system of claim 13, wherein the separation system comprises:
a quench tower that separates the cracked gas and the FCC product into an overhead product and a bottom product;
a fractionation tower that separates the bottom product into a fuel oil, a light cycle oil, and a slurry oil; and
an olefin recovery train that separates the overhead product into the plurality of hydrocarbon products.

19. The circular olefin system of claim 13, further comprising:
a pyrolysis unit that thermally treats a polyolefin to produce a pyrolysis product comprising the pyrolysis oil, wherein the pyrolysis unit is coupled to the fluid catalytic cracking unit.

20. The circular olefin system of claim 13, wherein the steam cracking unit does not crack naphtha hydrocarbons.
